(19) United States
(12) Patent Application Publication
(10) Pub. No.: US 2014/0271471 A1
(43) Pub. Date: Sep. 18, 2014

(54) THERMO-SENSITIVE BONE GROWTH COMPOSITIONS

(71) Applicant: Genzyme Corporation, Cambridge, MA (US)

(72) Inventors: Kuber T. Sampath, Holliston, MA (US); Michael Philbrook, Boston, MA (US); Aviva Shiedlin, Brookline, MA (US); John M. McPherson, Hopkinton, MA (US)

(73) Assignee: Genzyme Corporation, Cambridge, MA (US)

(21) Appl. No.: 14/209,059

(22) Filed: Mar. 13, 2014

Related U.S. Application Data
(60) Provisional application No. 61/783,803, filed on Mar. 14, 2013.

(51) Int. Cl. A61K 9/00; A61K 51/04; A61K 38/18

(52) U.S. Cl. CPC A61K 9/0024; A61K 38/1875; A61K 51/04
USPC 424/1.85; 514/8.8; 424/426

(57) ABSTRACT

A non-invasive injectable composition that contains type I collagen, an osteogenic growth factor (OSF), such as a bone morphogenetic protein and a reverse thermo-sensitive biodegradable polymer such as Poloxamer 407 in an aqueous vehicle. The formulation can be administered non-invasively, e.g., by injection, thus circumventing limitations of many currently marketed bone-inducing products. The injectable osteogenic formulation effectively induces bone formation at the desired locale. This injectable suspension could be used with bioresorbable bone mineral composites (e.g., Hydroxyapatite, Tri-calcium phosphate) and/or glycosaminoglycans (e.g., Hyaluronic acid, Heparin sulfate) to mold as putty and/slab as bone graft substitute implants to induce new bone formation in fracture healing and spine fusion procedures.

### CROSS-REFERENCE TO RELATED APPLICATIONS

This application claims priority to U.S. Application Ser. No. 61/783,803, filed on Mar. 14, 2013, which is hereby incorporated by reference.

### TECHNICAL FIELD

This invention relates to compositions to encourage bone growth and more particularly to bone graft substitutes (BGS) for inducing new bone formation.

### BACKGROUND

Surgical application of current BGS formulations, e.g., for fracture repair or spinal fusion, can be invasive, time-consuming and cumbersome due to the designs and configurations of the delivery systems that have been developed to deliver osteogenic growth factors.

### SUMMARY

We developed a non-invasive injectable composition that contains type I collagen, an osteogenic growth factor (OSF), and a reverse thermo-sensitive biodegradable polymer in an aqueous vehicle. The formulation can be administered non-invasively, e.g., by injection, thus circumventing limitations of many currently marketed bone-inducing products. The injectable osteogenic formulation effectively induces bone formation, as established, for example, by a standard rat model of ectopic bone formulation. The thermo-sensitive biodegradable polymer controls the rheology of the composition so that it can be injected at room temperature and, as its temperature increases to body temperature (37° C.), it forms a biocompatible gel that contains the OSF at delivery site, thus localizing the composition (and particularly the OSF) where it is useful. The use bone collagen powder in the composition provides an appropriate delivery matrix for the OSF and provides a biological environment that facilitates bone formulation. This injectable composition enables new bone formation at relatively low OSF concentrations.

In preferred embodiments of the composition, the OSF is a bone morphogenetic protein (BMP), such as BMP-2, BMP-4, BMP-6, BMP-7 (OP-1). Homodimers of BMP-2 or BMP-4 or BMP-6 or BMP-7 (OP-1) can be used, as can heterodimers of selected BMPs, such as a BMP-2/7 hetrodimer. Combination of selected BMPs may also be used. These proteins may be human proteins and they may be produced by recombinant means; they may be present at a concentration of less than 3.5 mg per g of thermo-sensitive collagen scaffold. The composition also may include a mineral such as tricalcium phosphate or hydroxylapatite. The composition may further include a bulking agent or visco supplement such as a hyaluronic compound, particularly one with a molecular weight >500Da. The hyaluronic compound may be cross-linked, such as cross-linked hyaluronic acid, to facilitate formation of molds or slabs at the implant site. A glycosaminoglycan such as chondroitin sulfate or chitosan may be included.

Also in preferred embodiments, at room temperature or below, the composition viscosity is suitable for injection from a syringe, e.g., the composition exhibits a syringe extrusion force ≦30 Newtons, when delivered from a 5 cc syringe with a needle size of 20 G-1.5″. The composition can be a malleable putty. The composition has between 50 and 80% liquid by weight. The average particle size of the collagen is between 70 and 425 μm, as determined by particle sieve. The composition components are dissolved/suspended in a buffered solution or sterile water. The composition can include a radio-contrast agent, and the composition need not include a hyaluronic compound.

The composition can be used to treat a patient in need of bone growth induction by injecting the composition at a site of desired bone growth, e.g. at a bone fracture site or, for a patient who is undergoing or has undergone a spinal fusion procedure, at the site of the spinal fusion

### DEFINITIONS

The term Hyaluronic Compound includes glycosaminoglycans (e.g., natural HA from living sources such as avian or bacterial sources, or synthetic HA), as well as hyaluronic acid salts and derivatives of the foregoing, including polymerized gels, cross-linked gels, and derivatized hyaluronic acid.

Osteogenic growth factor (OGF) means compounds that effect natural bone formation processes, such as Growth and Differentiation Factors (GDFs), Osteogenic Proteins (OPs), Osteoinductive Factors (OIFs). The term includes Bone Morphogenetic Proteins (BMP) such as BMP-2, BMP-4, BMP-6, BMP-7 (OP-1). In general these factors are well known and commercially available.

Poloxamers can be nonionic triblock copolymers composed of a central hydrophobic chain of polyoxypropylene (poly(propylene oxide)) flanked by two hydrophilic chains of polyoxyethylene (poly(ethylene oxide)). See generally U.S. Pat. No. 3,740,421. Poloxamers include the products Synperonics (Croda Inc., Edison N.J.), particularly poloxamer 407; Pluronic (BASFCorporation, Florham Park, N.J.); and Kolliphor (BASF Corporation, Tarrytown, N.Y.), a polyethoxylated castor oil and LeGoo® endovascular occlusion gel, which is comprised of a 20% (weight percent in saline) of purified poloxamer 407. Poloxamers are a family of biocompatible, water-soluble polymers that possess reverse, thermo-sensitive properties (i.e. as temperature increases, viscosity increases). In particular, the poloxamer used is non-toxic, biocompatible, water-soluble and its viscosity decreases with increasing temperature in a range of use. At room temperature the composition is injectable, but viscous. Upon heating to body temperature, it undergoes a temperature-induced phase change with no effective alteration in chemical composition—no curing—to form a polymeric plug or slab.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIG. 1 shows histological score of new bone formation: Evaluation of the ability of BMP-2, loaded on BBC in 20% Pluronic F-127, to induce bone formulation versus BMP-2 loaded on BBC in PBS (positive control); BBC, Bovine Bone Collagen.

FIG. 2A shows histological score of new bone formation: Effects of hyaluronic acid and Pluronic F-127 as added scaffold with BBC on the ability of BMP-2 to induce bone formation.

FIG. 2B shows histological scores for new bone formation in subcutaneous implants. Evaluation of various commercially available Hyaluronic acids as scaffold with BBC/Pluronic acid: Each dot represents an individual animal and the horizontal bar represents the group median score. There were no significant differences in group median scores for new bone/cartilage formulation between any of the groups (P>0.05).

FIG. 3 shows histological scores for new bone formulation in subcutaneous implants: Evaluation of radio-contrast agent (Isov) in combination with BBC/22. % Pluronic acid): Each dot represents an individual animal, and the horizontal bar represents the group median score. Key: +P<0.01 versus group 1 controls (22.5% PL+0 μg BMP-2); # #P<0.001 versus group 1 controls; ̂P<0.05 versus DGE group; PL=Pluronic.

FIG. 4 shows histological scores of new bone formation in subcutaneous implants: Evaluation of coral-hydroxyapatite scaffold in combination with BBC/Pluronic acid IBMP. Each circle represents an individual animal and the horizontal bar is the group median. Key: +P<0.01 and #P<0.001 versus 25 BBC+0μg BMP+P controls (group 1); P=Pluronic.

FIG. 4A shows histology scores for new bone formation in subcutaneous implants. Comparison of clinically used Bovine Achilles Tendon derived collagen (Heliostat-InFuse, Medtronic) with BBC/Pluronic acid scaffolds with various does of BMP-2. Each dot represents an individual animal and the horizontal bar represents the group median score. Key; I=Infuse®, BBC=bovine bone collagen/pluronate, ˜P<0.05 versus both 0 μg groups; A ̂P<0.01 versus both 0 μg groups; # P<0.001 versus both 0 μg groups; +P<0.01 versus H −5.4 μg group; ++ P<0.001 versus H −5.4 μg group, *P<0.05 vs H-5.4 μg group.

FIG. 5 shows pathology scores. Each circle represents an individual animal and the horizontal bar represents the group median score for new bone/cartilage production. There were no significant differences in group median scores between any of the groups receiving implants containing >1 μg BMP-2 (groups 2-12). Key: +P<0.001 versus group 1; *P<0.01 versus group 1; # P<0.05 versus group 1.

FIG. 6 shows the effect of BBC lot and scaffold size on alkaline phosphatase activity per gram of protein in the samples.

FIG. 7 shows the effects of different ratios of HA and Pluronic F-127 on alkaline phosphatase activity per gram of protein.

FIG. 8 shows the effects of different BBC lots and scaffold size on calcium concentration in the samples.

FIG. 9 shows the effect of different HA/Pluronic F-127 concentrations on calcium in the samples.

FIG. 10 shows the effect of different collagen scaffolds on the osteo-induction potential of BMP-2 in the rat ectopic model (22.5% Pluronic F-127 as a carrier).

FIG. 11 shows the effect of carriers on the osteo-induction potential of BMP-2 in the rat ectopic model (BBC, 70-425 um as scaffold).

FIG. 12 shows pathology scores for new bone/cartilage formation. Each dot represents an individual animal and the horizontal bar represents the group median score. There were no significant differences in group median scores for new bone/cartilage formation between any of the groups (P>0.05).

FIG. 13 shows calcium concentration in HA commercial products implants after 28 days.

FIG. 14 shows the osteoinduction potential of new batch of BBC, lot #17075-43, versus an existing batch of BBC, lot #11848-79, as scaffolds for BMP-2 in the rat ectopic model.

FIG. 15 shows an evaluation of carrier buffers, glutamate and PBS versus control, on the osteoinduction potential of BMP-2 in the rat ectopic model.

FIG. 16 is a comparison of two new carriers, 2.5% HA and 20% Pluronic F-127/2.5% HA with 20% Pluronic F-127.

FIG. 17 shows the effects of different carriers on the ability of rhBMP-2 to induce bone formation.

FIG. 18 is a comparison of two rat ectopic models: subcutaneous (SQ) versus intramuscular (IM) implantations in different carriers.

FIG. 19 is a scatter plot graph of pathology scores. Each dot represents an individual animal, and the horizontal bar represents the group median score. Key: +P<0.01 versus group 1 controls (22.5% PL+0 μg rhBMP-2); # P<0.001 versus group 1 controls; ̂P<0.05 versus DGE group; PL=pluronic.

FIG. 20 shows the effect of different carriers on calcium concentration.

FIG. 21 shows pathology scores for new bone/cartilage in implants. Each dot represents an individual animal and the horizontal bar represents the group median score. Symbols show statistical significance relative to control group 1. There were no significant differences between any of the treatment groups given >3 μg rhBMP-2 (P>0.05). Key: ̂P<0.05 versus group 1; +P<0.01 versus group 1; # P<0.001 versus group 1.

FIG. 22 shows the effects of RBC and BBC on calcium concentration.

FIG. 23 shows the effect of different BBC particle sizes, scaffold sizes and contrast agent in the carrier on calcium concentration.

FIG. 24 is a scatter plot graph of pathology scores for bone/cartilage production in the subcutaneous implants. Each dot represents an individual animal and the horizontal bar is the group median score. Key: +(P<0.05) vs BMP-4, 3 μg); # (P<0.05 vs BMP-4, 0.3 μg); * (p<0.01 vs BMP-4, 3 μg); ̂(P<0.001 vs BMP-4, 3 μg and 0.3 μg); a (P<0.05 vs BMP-4, 1 μg); b (P<0.01 vs BMP-4, 0.3 μg); PL=22.5% pluronic; sqi=subcutaneous injection.

### DETAILED DESCRIPTION

In general the following protocol illustrates formation of an injectable osteogenic composition. The OGF, type I collagen, and polymer component are simply illustrative and those in the art will understand that for clinical use the components will be selected from those which are approved for human clinically use.

Osteogenic Solution Preparation:

Sterile, insoluble and particulate bovine bone-derived Type 1 collagen was taken up in 30-40% ethanol (v/v in water) in 0.01 N HCl and to which the BMP solution in 0.01 N HCl or in glutamate buffer (pH 4.8) was added aseptically, vortexed ×3, incubated at 4° C. for an hr, and then subjected to lyophilization. The amount of BMP added in the concentrations is in the range of 1-100 μg to 25-200 mg of collagen. The lyophilized collagen/BMP matrix (˜25 mg) was then taken up in 150 μl of 20-40% pluronic polymer (v/v) in PBS (pH 7.4) and mixed thoroughly at room temperature (RT) for injection. In some instances, collagen-BMP-pluronic mixture was combined with a bulking agent (e.g. hyaluronic acid, bone mineral or combination thereof). Alternately, BMP-Bone Collagen-Pluronic Polymer, Mineral and Glycosaminao Glycans with radio contrast agent mixture can be lyophilized in a sterile environment and can be suspended in water or buffered solution prior to use at the operation suites.

Bone Induction Assay:

The bone-inducing activity of the injectable osteogenic formulation can be assessed by implantation at subcutaneous sites or by injecting percutaneously into abdominal fascia or skeletal muscle pouches of rodents. At 12-21 days after injections, the implants were harvested, and assayed for bone forming activity by biochemical analyses (alkaline phosphatase and calcium content) and histology as described (Sampath. T. K. and Reddi, A. H. 1981).

OGF:

The OGF, e.g. natural or recombinant human BMP, such as BMP-2 or BMP-7 (OP-1) or BMP-4 or BMP-6, or mixtures can be obtained from the commercial sources.

Collagenous Matrix:

Type I collagen can be obtained from numerous commercial sources. The examples below use Bovine bone Type I. collagen is prepared as described (Sampath. T. K. and Reddi, A. H. 1981). In clinical use, the type I collagen should be one that can be used in treating humans.

Bovine demineralized diaphyseal bone matrix, DBM (70-420 μm) as prepared from 3-6 months old cows by using standard procedures. The bovine DBM was then subjected 6 M guanidine HCl at 4° C. for several hrs (16-24 hrs) and then washed with water, heated for 1 hr in acidic environment and then water washed and ethanol treated prior to lyophilization. Demineralized, insoluble, gguanidine-HCl extracted and acid treated bovine bone type I collagen was sterilized by subjecting to 3.5 mega RAD gamma radiation prior to use, then subjected to sterile water wash with free radical scavengers and lyophilzation.

Hyaluronic Acid (HA) Products:

Bacterially derived HA (average molecular weight 3,000,000) was purified by fermentation of Streptococcus zooepidemicus in Genzyme facility in Framingham, Mass. Hylan A (average molecular weight 6,000,000) produced from chicken combs at the Genzyme facility in Ridgefield, N.J.

Prevelle Silk and Dermal Gel Extra (DGE) are dermal filers. They were prepared at the Genzyme facility in Ridgefield, N.J.

Hylastan is a visco-supplement to be used for the treatment of pain due to osteoarthritis. It was prepared at the Genzyme facility in Ridgefield. N.J. Restylane is a dermal filler and was purchased from QMED, Sweden.

Properties of Commercial Products of Hyaluronic Acid (HA)

| | (DGE) | Prevelle Silk | Restylane* | Hylastan/ |
|---|---|---|---|---|
| Total HA Concentration (mg/mL) | 22 | 5.5 | 20 | 10 |
| Gel/Fluid Ratio | 85/15 | 98/2 | 75/25 | 65/35 |
| HA Gel Concentration (mg/mL) | 18.7 | 5.4 | 15.0 | 10 |
| Degree of HA Modification (%) | 9 | 23 | 3 | 3.5 |
| % Cross-linked HA | 7 | 12 | 1.2 | 2.7 |
| Dilution Durability/% Swelling | ~50 | <25 | 50 | |
| G′ Modulus (Pa) | 1800 | 230-260 | 660 | |
| Average Particle Size (micron) | 230 | 350 | 300 | |
| Chemistry | DVS | DVS | BDDGE | DVS |
| HA Fluid MW [kDa] - Pre | — | — | — | 3MD |
| sterilisation | | | | |
| Lidocane | + | + | − | − |
| In vivo residence time [month] | 9 | 4-6 | 6-9 | ~1-2 |
| G needle | 30 | 31 | 30 | 18-21 |
| Extrusion force (1 ml/G30) [N] | 33 | 18* | 27 | |

Poloxamer 407 Polymer:

Poloxamer 407/Pluronic F-127 copolymer (ethylene oxide and propylene oxide blocks) was purchased from BASF (Mount Olive, N.J.).

The polymer was solubilized in PBS for a final polymer concentration of 20-30% wt/volume. At this concentration the polymer shows thermo-reversible properties, fluid state at room temperature and gel state at body temperature. 20% gels were prepared by adding 20 g of Pluronic F-127 to 100 mL of cold PBS and left under agitation overnight at 4° C. for proper solubilization. The solution was next filtered with a 0.22 μm filter for sterilization.

Characteristics of Composition:

The composition has a viscosity and an extrusion force that enable its use in a syringe. For example, it is delivered from a 5 cc syringe with a needle size of 20 G-1.5″ with an extrusion three of less than 30 Newtons.

### EXAMPLES

### Example 1

The injectable osteogenic formulation induced endochondral bone formation as judged by the alkaline phosphatase activity, calcium content and histological evaluation of sample explants from the rat model of ectopic bone formation. The level of bone-inducing activity was dependent BMP protein concentration (FIG. 1).

### Example 2

Poloxamer 407 concentrations ranging from 20-30% were examined in these studies. While all Poloxamer-containing formulations induced one formation, the optimal concentration of Poloxamer 407 observed is 20-25% to accelerate gel formation in vivo (FIG. 2A).

### Example 3

Percutaneous injection of collagen-BMP matrix with high molecular weight hyaluronic acid (Hyal-A or Hyalastin) solution with or without plutonic acid also induced new bone formulation (FIG. 2B).

### Example 4

Since clinical use of the injectable osteogenic formulation may require fluoroscopic guidance to the intended site of delivery, we demonstrate that addition of radio-contrast agent to the formulation did not interfere with bone formulation in vivo. The injectable osteogenic formulation was supplemented with a clinically relevant concentration of radio-contrast agent (e.g. Isovue-370) and tested the rat model of ectopic bone formulation. Results of this study revealed that radio-contrast agent did not interfere with bone induction in this animal model of bone formation (FIG. 3).

### Example 5

Since coral-derived hydroxyapatite has been used as bone avoid filler and bulking mineral scaffold with autologous bone graft, we examined, the effect of ProOsteon® 500R (Interporc, Cross International) for new bone formation. The results suggest that coral-hydroxyapatite is biocompatible with BBC/Plutonic acid and forms as moldable putty to use as bone graft substitute for spine fusion (FIG. 4).

### Example 6

InFuse (Medtronic, Minn.) has been approved for use inter-body fusion for lumbar spine. Infuse employs 12 mg of BMP-2 soaked with the sheet of bovine Achilles tendon derived type I collagen, and threaded into the pocked of titanium metal cage to stimulate new bone formulation and fuse the adjacent segments of lumbar bar spine. We have compared the InFuse-bovine Achilles collagen with BBC/Pluronic injectable suspension with various doses of BMP-2 subcutaneous implants. The results show that BBC/Pluronic acid suspension employs 10-50 times less BMP-2 for given volume of collagen implants to elicit comparable new bone formulation as evidenced, by histological scores (FIG. 5).

### Example 7

### 09-4662

This example investigates
the osteo-inductive potential of collagen carrier lots in a rat ectopic model
collagen/BMP ratios and the effect of scaffold mass on osteo-induction potential
variations in carrier/polymer ratio on osteo-induction in rat ectopic model

Study Design Twelve groups (n=4) of 4-5 week old male Long Evans rats received bilateral subcutaneous implants in the chest. Surgical implants contained 0-10 μg BMP-2 (signal) in varying concentrations of HA/Pluronic F-127 (carrier) and varying amounts of BBC (scaffold).

Details of the study design are outlined in Table 1 below.

All rats were sacrificed via CO₂ asphyxiation on day 14 and samples were harvested for analysis.

TABLE 1
Study Design

| Group | BBC | | BMP-2 Dose | Carrier [% wt/wt] | | Vol. |
|---|---|---|---|---|---|---|
| # | [mg]/implant | [% wt/wt] | [μg] | HA | F-127 | [μL] |
| 1 | 25* | 14.3 | 0 | 0 | 16 | 150 |
| 2 | 25* | 14.3 | 1 | 0 | 16 | 150 |
| 3 | 25* | 14.3 | 3 | 0 | 16 | 150 |
| 4 | 25 ** | 14.3 | 3 | 0 | 16 | 150 |
| 5 | 50* | 25 | 6 | 0 | 16 | 150 |
| 6 | 40* | 40 | 5 | 0 | 16 | 100 |
| 7 | 40* | 40 | 5 | 1.5 | 12 | 100 |
| 8 | 40* | 40 | 5 | 4.3 | 4.3 | 100 |
| 9 | 40* | 40 | 5 | 6 | 1.5 | 100 |
| 10 | 40* | 40 | 10 | 1.5 | 12 | 100 |
| 11 | 40* | 40 | 10 | 4.3 | 4.3 | 100 |
| 12 | 40* | 40 | 10 | 6 | 1.5 | 100 |

*Lot # 18034-124
** Lot #18034-104

Each sample was cut into two pieces. One half of the sample was fixed in 10% neutral buffered formalin, embedded in methylmethacrylate, sectioned at approximately 5 microns and stained with hematoxylin and eosin (H&E), von Kossa, and toluidine blue. Histopathologic evaluation included qualitative and semi-quantitative assessment of new cartilage and bone formation in the samples, using the scoring system outlined in Table 2. The distribution pattern of new bone/cartilage formation was also scored for each sample (Table 3) (Lucy Phillips, B.V.Sc., A.C.V.P, Pathology Department, Genzyme Corporation).

TABLE 2
Scoring system for new bone/cartilage

| Score | Description |
|---|---|
| 0 | No new bone |
| 1 | 1-25% of the implant contains new bone or cartilage |
| 2 | 25-50% of the implant contains new bone or cartilage |
| 3 | 50-75% of the implant contains new bone or cartilage |
| 4 | 75-100% of the implant contains new bone or cartilage |

TABLE 3
Distribution pattern of new bone/cartilage

| Pattern | Description |
|---|---|
| A | Focal; new bone forms a rim around a cavitated center |
| B | Focal; new bone forms a rim around proliferating |
| | mesenchymal cells |
| C | Focal; new bone extends throughout the implant in |
| | a solid pattern |
| D | Multifocal; new bone forms a rim around multiple |
| | cavitated areas |
| E | Multifocal; new bone forms a rim around multiple |
| | foci of proliferating mesenchymal cells |
| F | Multifocal; several nodules of new bone extend |
| | throughout the implant in a solid pattern |

The other half of sample was cleaned of adherent tissue. The sample was placed in 2 ml of ice-cold 0.15 M NaCl/3 mM NaHCO₃ and then homogenized using a Polytron homogenizer. It was then centrifuged; the supernatant was decanted and analyzed for total protein concentration (TP) and alkaline phosphatase activity (ALP) by Randox Daytona chemical analyzer. (Michelle Searles, Department of harmacology/Toxicology; Genzyme Corporation).

The residue was washed twice in 5 ml of 20 mM phosphate buffer, and then extracted in 5 ml of 0.6 NHCL overnight at 4° C. It was then centrifuged; the supernatant was decanted and sent for calcium analysis. Samples were analyzed on a Varian ICP-OES at 396 nm emission by Martin Hanus, Department of Analytical Research and Development; Genzyme Corporation.

Results: Histopathologic Evaluation.

The scatter plot graph of pathology scores is shown in FIG. 5.

The degree and distribution pattern of new bone/cartilage was comparable in samples containing 25 or 50 mg of BBC, lot #18034-124, and in samples containing lot #18034-124 (new) or lot #10834-104 (old).

For samples containing varying concentrations of HA/Pluronic F-127 carrier, there were no differences in the degree of new bone production; however, there was a noticeable trend for new bone to form in a rim around a cavitated center containing BBC scaffold and hemorrhage as the HA concentration increased.

Alkaline phosphatase activity
For implants containing 3 μg BMP-2, there were no differences in alkaline phosphatase concentration
Increasing the BBC scaffold from 25 to 50 mg (with the same BBC/BMP-2 ratio) did not result in higher alkaline phosphatase concentration
At 5 μg BMP-2, increasing the concentration of HA and reducing the concentration of Pluronic F 127 in the carrier did not result in differences in alkaline phosphatase concentration
At 10 μg BMP-2 load, samples with higher HA concentration (6%) had higher alkaline phosphatase concentration, but not above the variation in the assay

### Calcium Evaluation

For implants containing 3 μg BMP-2, there was a slight trend for higher Ca content for the old lots of BBC (lot #10834-104) compared to the new lot (lot #10834-124)
Increasing the BBC scaffold from 25 to 50 mg (with the same BBC/BMP-2 ratio) did not result in higher Ca content
At 5 μg of BMP-2, increasing the concentration of HA and reducing the concentration of Pluronic F-127 in the carriers did not result in any differences in Ca concentration
Samples with 10 μg BMP-2 and 1.5% F-127/6% HA carrier had 25% higher Ca concentration then the others formulations tested.

### CONCLUSIONS

The degree and distribution pattern of new bone/cartilage, calcium and alkaline phosphatase concentrations were comparable in implants containing 25 and 50 mg of BBC, and in implants containing lot #10834-124 and lot #10834-104 BBC scaffolds
For implants containing varying concentrations of HA and Pluronic F-127 in a carrier, there were no differences in the degree of new bone production; however, as the HA concentration increased, there was a noticeable trend for new bone to form as a rim around a cavitated center containing BBC scaffold and hemorrhage

### Example 8

### 09-3467

This example investigates
The effect of cartilage particle size
Effect of polymer concentration in the carrier on osteo-induction
Effect of carrier/polymer ratio
Effect of contrast agent

Study Design Twelve groups of 4-5 week old male Long Evans rats received bilateral subcutaneous injection in the chest.

Three groups received surgical implants containing 5 μg BMP-2 (signal) loaded on 25 mg of bovine bone collagen (BBC, lot #17075-43, scaffold) with different ratios of contrast agent (Isovue-370)/Pluronic F-127 as a carrier (groups 1-3). Eight groups received surgical implants containing 5 μg BMP-2 (signal) loaded on 25 mg of varying collagens (scaffold) in Pluronic F-127 (carrier) in PBS. Collagens used in this study include the following:
BBC, lot #17075-43, 70-425 um
BBC, lot #17075-128, 70-250 um
Soluble collagen (MP Biologics)
Febrile collagen (Instant MCH, Ethicon)

Two groups received surgical implants containing 5 μg BMP-2 (signal) loaded on 25 mg of BBC (lot #17075-43) scaffold, in 20% Pluronic F-127/2.5% HA or Dermal Gel Extra (DGE) carriers.

All rats were sacrificed via CO₂ asphyxiation on day 14 and samples were harvested for analysis.

Details of the study design are outlined in Table 4 below.

TABLE 4
Study Design

| | | BMP-2 Dose | | # |
|---|---|---|---|---|
| Group # | Scaffold | [μg] | Carrier | Animals/Group |
| 1 | BBC, 70-425 um | 5 | Pluronic F-127/20% Isovue 370 | 4 |
| 2 | BBC, 70-425 um | 5 | Pluronic F-127/40% Isovue 370 | 4 |
| 3 | BBC, 70-425 um | 5 | Pluronic F-127/80% Isovue 370 | 4 |
| 4 | BBC, 70-425 um | 5 | 22.5% Pluronic F-127 | 4 |
| 5 | BBC, 70-425 um | 1 | 22.5% Pluronic F-127 | 4 |
| 6 | BBC, 70-425 um | 0 | 22.5% Pluronic F-127 | 4 |
| 7 | Soluble collagen | 5 | 22.5% Pluronic F-127 | 6 |
| 8 | Febrile collagen | 5 | 22.5% Pluronic F-127 | 4 |
| 9 | BBC, 70-250 um | 5 | 22.5% Pluronic F-127 | 4 |
| 10 | BBC, 70-250 um | 1 | 22.5% Pluronic F-127 | 4 |
| 11 | BBC, 70-425 um | 5 | DGE | 4 |
| 12 | BBC, 70-425 um | 5 | 2.5% HA/20% Pluronic F-127 | 4 |

The implants were harvested, fixed in 40% alcohol, embedded in methylmethacrylate, sectioned at approximately 5 microns and stained with hematoxylin and eosin (H&E) and toluidine blue.

Histopathologic analysis was performed by Kuber Sampath (Genzyme) and included semi-quantitative assessment of new bone production in the implant, using the scoring system outlined in Table 5.

TABLE 5
Scoring system for new bone

| Score | Description |
|---|---|
| 0 | No new bone |
| 1 | 1-20% of the implant area contains new bone |
| | (w/o area occupied by BBC) |
| 2 | 21-40% of the implant area contains new bone |
| | (w/o area occupied by BBC) |
| 3 | 41-60% of the implant area contains new bone |
| | (w/o area occupied by BBC) |
| 4 | 61-80% of the implant area contains new bone |
| | (w/o area occupied by BBC) |
| 5 | 81-100% of the implant area contains new bone |
| | (w/o area occupied by BBC) |

Results: Implants with febrile collagen scaffold could not be identified at the time of harvest. Therefore no samples were taken (group 8).

Data is presented in Table 6 and FIGS. 10 and 11.

TABLE 6
Histology scores

| Group | | BMP-2 | | Histology Score | |
|---|---|---|---|---|---|
| # | Scaffold | Dose [μg] | Carrier | Average | STDEV |
| 1 | BBC, 70-425 um | 5 | Pluronic F-127/20% Isovue 370 | 4.3 | 0.5 |
| 2 | BBC, 70-425 um | 5 | Pluronic F-127/40% Isovue 370 | 4.3 | 0.5 |
| 3 | BBC, 70-425 um | 5 | Pluronic F-127/80% Isovue 370 | 3.5 | 1.0 |
| 4 | BBC, 70-425 um | 5 | 22.5% Pluronic F-127 | 4.4 | 1.0 |
| 5 | BBC, 70-425 um | 1 | 22.5% Pluronic F-127 | 3.4 | 1.0 |
| 6 | BBC, 70-425 um | 0 | 22.5% Pluronic F-127 | 0.0 | 0.0 |
| 7 | Soluble collagen | 5 | 22.5% Pluronic F-127 | 1.0 | 0.5 |
| 8 | Febrile collagen | 5 | 22.5% Pluronic F-127 | 0.0 | 0.0 |
| 9 | BBC, 70-250 um | 5 | 22.5% Pluronic F-127 | 4.3 | 1.0 |
| 10 | BBC, 70-250 um | 1 | 22.5% Pluronic F-127 | 3.9 | 0.5 |
| 11 | BBC, 70-425 um | 5 | DGE | 3.3 | 0.5 |
| 12 | BBC, 70-425 um | 5 | 22.5% Pluronic F-127/HA | 3.9 | 1.0 |

For the 1 μg rhBMP-2 dose, implants containing 25 mg of the smaller particle BBC showed a trend for greater new bone production relative to the dose-equivalent large particle BBC group.
For the 5 μg rhBMP-2 dose, BBC implants with different particle size showed comparable new bone production.
Soluble collagen showed very poor bone production compare to both lots of BBC.
Addition of 20 or 40% Isovue to the 22.5% Pluronic F-127 carrier had no effect on osteoinduction potential.
When 80% Isovue was added to the Pluronic F-127, new bone/cartilage production in implants trended lower.
When HA was added to the Pluronic F-127, or when DGE was used as the sole carrier, new bone/cartilage production in implants trended lower.

### CONCLUSION

BBC particle size had no effect on BMP-2 osteoinduction potential.
Soluble collagen had very poor new bone production.
Addition of Isovue to the 22.5% Pluronic F-127 carrier had no effect on osteoinduction potential.
When HA was added to the Pluronic F-127, or when DGE was used as the sole carrier, new bone/cartilage production in implants was lower

### Example 9

### 09-3468

This example investigates
BMP dose response with and without polymer
Osteoinduction by recombinant BMPs
Varying the carrier

Study Design: Thirteen groups (n=4/group) of 4-5 week old male Long Evans rats received bilateral subcutaneous implants in the chest.

Eight groups received surgical implants contained 10 μg BMP-2 (signal) loaded on 25 mg of bovine bone collagen (BBC, lot #17075-43, scaffold) and formulated with different hyaluronic acid (HA) commercial products (carrier).

Five groups received surgical implants containing 3-10 μg BMP-2 or BMP-4 loaded on 25 mg of BBC with Pluronic® F.-127.

All rats were sacrificed via CO₂ asphyxiation on days 14 or 28 and samples were harvested for analysis.

Details of the study design are outlined in Table 7 below.

TABLE 7
Study Design

| | | | | Resi- |
|---|---|---|---|---|
| | | | | dence |
| Group | Scaf- | | | time |
| # | fold | Signal | Carrier | [weeks] |
| 1 | BBC | BMP-2, 10 μg | DGE | 2 |
| 2 | BBC | BMP-2, 10 μg | DGE | 4 |
| 3 | BBC | BMP-2, 10 μg | Prevelle Silk | 2 |
| 4 | BBC | BMP-2, 10 μg | Prevelle Silk | 4 |
| 5 | BBC | BMP-2, 10 μg | Hylastan | 2 |
| 6 | BBC | BMP-2, 10 μg | Hylastan | 4 |
| 7 | BBC | BMP-2, 10 μg | Restylane | 2 |
| 8 | BBC | BMP-2, 10 μg | Restylane | 4 |
| 9 | BBC | BMP-2, 3 μg | 22.5% Pluronic F-127 in PBS | 2 |
| 10 | BBC | BMP-2, 3 μg | 22.5% Pluronic F-127 in PBS | 4 |
| 11 | BBC | BMP-4, 10 μg | 22.5% Pluronic F-127 in glut | 2 |
| 12 | BBC | BMP-4, 5 μg | 22.5% Pluronic F-127 in glut | 2 |
| 13 | BBC | BMP-2, 5 μg | 22.5% Pluronic F-127 in glut | 2 |

Evaluation:

Samples were collected from each test site at the time of necropsy (14 or 28 days). Each sample was cut into two pieces. One half of the sample was fixed in 10% neutral buffered formalin, embedded in methylmethacrylate, sectioned at approximately 5 microns and stained with hematoxylin and eosin (H&E), von Kossa, and toluidine blue. Histopathologic evaluation included qualitative and semi-quantitative assessment of new cartilage and bone formation in the samples and used the scoring system outlined in Table 8. The distribution pattern of new bone/cartilage formation was also scored for each sample (Table 9) (Lucy Phillips, B.V.Sc., A.C.V.P, Pathology Department, Genzyme Corporation).

TABLE 8
Scoring system for new bone/cartilage

| Score | Description |
|---|---|
| 0 | No new bone |
| 1 | 1-25% of the implant contains new bone or cartilage |
| 2 | 25-50% of the implant contains new bone or cartilage |
| 3 | 50-75% of the implant contains new bone or cartilage |
| 4 | 75-100% of the implant contains new bone or cartilage |

TABLE 9
Distribution pattern of new bone/cartilage

| Pattern | Description |
|---|---|
| A | Focal; new bone forms a rim around a cavitated center |
| B | Focal; new bone forms a rim around proliferating |
| | mesenchymal cells |
| C | Focal; new bone extends throughout the implant in |
| | a solid pattern |
| D | Multifocal; new bone forms a rim around multiple |
| | cavitated areas |
| E | Multifocal; new bone forms a rim around multiple |
| | foci of proliferating mesenchymal cells |
| F | Multifocal; several nodules of new bone extend |
| | throughout the implant in a solid pattern |

The other half of sample was cleaned of adherent tissue. The sample was placed in 2 ml of ice-cold 0.15 M NaC13 mM NaHCO₃ and then homogenized using a Polytron homogenizer. It was then centrifuged; the supernatant was decanted.

The residue was washed twice in 5 ml of 20 mM phosphate buffer, and then extracted in 5 ml of 0.6 NHCL overnight at 4° C. It was then centrifuged; the supernatant was decanted and sent for calcium analysis. Samples were analyzed on a Varian ICP-OES; at 396 nm emission (Martin Hanus, Department of Analytical Research and Development; Genzyme Corporation).

Results:

At the time of necropsy at 14 days, all implants with BMP-4 (groups 11 & 12) could not be identified and therefore no samples were taken.

At the time of necropsy at 28 days, all implants with 22.5% Pluronic F-127 (group 10) could not be identified and therefore no samples were taken.

### Histopathologic Evaluation

The scatter plot graph of pathology scores for HA products at days 14 and 28 are shown in FIG. 12.
All groups had comparable median scores for new bone/cartilage formation in the implants at day 14 or 28, irrespective of the HA carrier used (P>0.05)
There was no consistent distribution pattern of new bone/cartilage in any of the groups

### Calcium Evaluation

Implants with DGE and Prevelle Silk had a trend for higher % Ca concentrations compared to Hylastan and Restylane.

### CONCLUSIONS

At 14 days implants with BMP-4 could not be identified
At 28 days implants with 22.5% Pluronic F-127 were completely reabsorbed
At 14 and 28 days, implants with rhBMP-2, BBC and HA commercial products had comparable new bone/cartilage formation irrespective of the HA carrier used
At day 28, implants containing rhBMP-2 and HA commercial products loaded scaffolds, had comparable Ca concentration irrespective of the HA carrier used

### Example 10

### 08-3212

This example investigates
the osteo-inductive potential of collagen carrier lots in a rat ectopic model
Effect of polymer concentration in the carrier on osteo-induction
Effect of carrier/polymer ratio

Study Design: Sixteen groups of male 6 weeks old Long Evans rats were used in this study. Test articles were surgically implanted in the subcutaneous pockets bilaterally in the chest
Two groups received implants containing 1.5 or 5 μg BMP-2 (signal) loaded on 25 mg of BBC (lot #17075-43, scaffold) in 150 μl 20% Pluronic F-127 in glutamate buffer (carrier) (groups 1 &2)
Four groups received implants containing 0-5 μg BMP-2 (signal) loaded on 25 mg of BBC (lot #17075-43, scaffold) in 150 μl 20% Pluronic F-127 in PBS (carrier) (groups 3-6)
Two groups received implants containing 1.5 or 5 μg BMP-2 (signal) loaded on 25 mg of BBC (lot #17075-43, scaffold) in 150 μl 20% Pluronic F-127+2.5% HA in glutamate buffer (carrier) (groups 7&8)
Four groups received implants containing 0-5 μg BMP-2 (signal) loaded on 25 mg of BBC (lot #17075-43, scaffold) in 40 μl PBS (positive control) (groups 9-12).
Three groups received implants containing 0-5 μg BMP-2 (signal) loaded on 25 mg of BBC (lot #11848-79, scaffold) in 150 μl 20% Pluronic F-127 in PBS (carrier) (groups 13-15)
One group received implants containing 1.5 μg BMP-2 (signal) loaded on 25 mg of BBC (lot #17075-43, scaffold) in 150 μl 2.5% HA in glutamate buffer (carrier) (group 16)
   Rats were sacrificed on day 14 post-implantation via CO₂ asphyxiation and the implants were harvested.

Details of the study design are outlined in Table 10 below.

TABLE 10
Study Design

| | | BMP-2 | | | # |
|---|---|---|---|---|---|
| Group | Scaf- | Dose | | Volume | Ani- |
| # | fold | [μg] | Carrier | [μl] | mals |
| 1 | BBC* | 5.0 | 20% Pluronic F-127 in glut. | 150 | 3 |
| 2 | BBC* | 1.5 | 20% Pluronic F-127 in glut. | 150 | 3 |
| 3 | BBC* | 5.0 | 20% Pluronic F-127 in PBS | 150 | 4 |
| 4 | BBC* | 1.5 | 20% Pluronic F-127 in PBS | 150 | 3 |
| 5 | BBC* | 0.5 | 20% Pluronic F-127 in PBS | 150 | 3 |
| 6 | BBC* | 0.0 | 20% Pluronic F-127 in PBS | 150 | 3 |
| 7 | BBC* | 5.0 | 20% Pluronic F-127 + | 150 | 3 |
| | | | 2.5% HA in glut. | | |
| 8 | BBC* | 1.5 | 20% Pluronic F-127 + | 150 | 3 |
| | | | 2.5% HA in glut. | | |
| 9 | BBC* | 5.0 | Control, PBS | 40 | 3 |
| 10 | BBC* | 1.5 | Control, PBS | 40 | 3 |
| 11 | BBC* | 0.5 | Control, PBS | 40 | 3 |
| 12 | BBC* | 0.0 | Control, PBS | 40 | 3 |
| 13 | BBC** | 5.0 | 20% Pluronic F-127 in PBS | 150 | 4 |
| 14 | BBC** | 1.5 | 20% Pluronic F-127 in PBS | 150 | 3 |
| 15 | BBC** | 0.0 | 20% Pluronic F-127 in PBS | 150 | 3 |
| 16 | BBC* | 1.5 | 2.5% HA in glut. | 150 | 3 |

*BBC, lot # 17075-43
**BBC, lot # 11848-79

Evaluation:

The implants were harvested, fixed in 40% alcohol, embedded in methylmethacrylate, sectioned at approximately 5 microns and stained with hematoxylin and eosin (H&E) and toluidine blue.

Histopathologic analysis was performed by Kuber Sampath (Genzyme) and included semi-quantitative assessment of new bone production in the implant, using the scoring system outlined in Table 2.

TABLE 11
Scoring system for new bone production

| Score | Description |
|---|---|
| 0 | No new bone |
| 1 | 1-20% of the implant area contains new bone |
| | (w/o area occupied by BBC) |
| 2 | 21-40% of the implant area contains new bone |
| | (w/o area occupied by BBC) |
| 3 | 41-60% of the implant area contains new bone |
| | (w/o area occupied by BBC) |
| 4 | 61-80% of the implant area contains new bone |
| | (w/o area occupied by BBC) |
| 5 | 81-100% of the implant area contains new bone |
| | (w/o area occupied by BBC) |

Results:

Data is presented in Table 12 and FIGS. 14, 15 and 16.

TABLE 12
Table of histology scores

| Group | | BMP-2 | | Histology Score | |
|---|---|---|---|---|---|
| # | Scaffold | Dose [μg] | Carrier | Average | STDEV |
| 1 | BBC* | 5.0 | 20% Pluronic F-127 in glut. | 4.6 | 0.5 |
| 2 | BBC* | 1.5 | 20% Pluronic F-127 in glut. | 3.5 | 0.5 |
| 3 | BBC* | 5.0 | 20% Pluronic F-127 in PBS | 3.8 | 0.8 |
| 4 | BBC* | 1.5 | 20% Pluronic F-127 in PBS | 4.3 | 0.5 |
| 5 | BBC* | 0.5 | 20% Pluronic F-127 in PBS | 1.5 | 1.6 |
| 6 | BBC* | 0.0 | 20% Pluronic F-127 in PBS | 0.0 | 0.0 |
| 7 | BBC* | 5.0 | 20% Pluronic F-127 + 2.5% HA in glut. | 3.0 | 2.3 |
| 8 | BBC* | 1.5 | 20% Pluronic F-127 + 2.5% HA in glut. | 1.6 | 2.5 |
| 9 | BBC* | 5.0 | PBS | 4.6 | 0.5 |
| 10 | BBC* | 1.5 | PBS | 4.3 | 0.5 |
| 11 | BBC* | 0.5 | PBS | 3.8 | 0.8 |
| 12 | BBC* | 0.0 | PBS | 0.0 | 0.0 |
| 13 | BBC** | 5.0 | 20% Pluronic F-127 in PBS | 3.8 | 0.8 |
| 14 | BBC** | 1.5 | 20% Pluronic F-127 in PBS | 1.6 | 1.5 |
| 15 | BBC** | 0.0 | 20% Pluronic F-127 in PBS | 0.0 | 0.0 |
| 16 | BBC* | 1.5 | 2.5% HA in glut. | 3.0 | 2.4 |

*BBC, lot # 17075-43
**BBC, lot # 11848-79

For the 5 μg rhBMP-2 dose, implants with both BBC batches showed comparable osteoinduction potential. For the 1.5 μg rhBMP-2 dose, the new batch of BBC (lot #17075-43), showed greater osteoinduction potential.

For all rhBMP-2 doses, two buffers showed comparable osteoinduction potential. Implants with 2.5% HA and 20% Pluronic F-127/2.5% HA, had variable histology scores and were smaller than in the 20% Pluronic F-127 group.

### CONCLUSION

The two batches of BBC tested had comparable osteoinduction potential at a 5 μg BMP-2 dose.
20% Pluronic F-127/2.5% HA and 2.5% HA had lower osteo-induction potential than 20% Pluronic F-127.
Two buffers, glutamate buffer and PBS showed comparable osteoinduction potential.

### Example 11

### 09-2764

Study Objectives:
To evaluate different concentrations of OGF carriers

Study Design

Twelve groups of 4-5 week old male Long Evans rats received bilateral subcutaneous implants in the chest. Surgical implants volume was held constant at 250 μl and used BMP-2 at 5 μg dose (signal). Carriers were tested in the presence or absence of BBC. Rats were sacrificed on day 14 post-implantation via CO₂ asphyxiation and the implants were harvested.

Details of the study design are outlined in Table 13 below.

TABLE 13
Study Design

| | | BMP-2 Dose | Implant | # |
|---|---|---|---|---|
| Group # | Scaffold | [μg] | Carrier | Animals/Group |
| 1 | BBC | 5 | Glutamate Buffer | 5 |
| 2 | — | 5 | 2.5% HA | 4 |
| 3 | BBC | 5 | 2.5% HA | 6 |
| 4 | — | 5 | 5% HA | 3 |
| 5 | BBC | 5 | 5% HA | 3 |
| 6 | BBC | 5 | 2.5% Hylan-A | 3 |
| 7 | — | 5 | 2.5% Hylan-A | 3 |
| 8 | BBC | 5 | 5% Hylan-A | 3 |
| 9 | — | 5 | 30% Pluronic F-127 | 3 |
| 10 | BBC | 5 | 30% Pluronic F-127 | 3 |
| 11 | — | 5 | 20% Pluronic F-127 | 3 |
| 12 | BBC | 5 | 20% Pluronic F-127 | 3 |

Evaluation:

The implants were harvested, fixed in 40% alcohol, embedded in methylmethacrylate, sectioned at approximately 5 microns and stained with hematoxylin and eosin (H&E) and toluidine blue.

Histopathologic analysis was performed by Kuber Sampath (Genzyme) and included semi-quantitative assessment of new bone production in the implant, using the scoring system outlined in Table 14.

TABLE 14
Scoring system for new bone production

| Score | Description |
|---|---|
| 0 | No new bone |
| 1 | 1-20% of the implant area contains new bone |
| | (w/o area occupied by BBC) |
| 2 | 21-40% of the implant area contains new bone |
| | (w/o area occupied by BBC) |
| 3 | 41-60% of the implant area contains new bone |
| | (w/o area occupied by BBC) |
| 4 | 61-80% of the implant area contains new bone |
| | (w/o area occupied by BBC) |
| 5 | 81-100% of the implant area contains new bone |
| | (w/o area occupied by BBC) |

Results:

Samples that did not contain BBC (groups 2, 4, 7, 9 and 11) and samples that did not contain a carrier (group 1) could not be identified at tissue harvest, therefore no samples were collected for these groups.

Pathology scores are presented in Table 15 and FIG. 17.

TABLE 15
Table of histology scores

| Group | | BMP-2 | | Histology Score | |
|---|---|---|---|---|---|
| # | Scaffold | Dose [μg] | Carrier | Average | STDEV |
| 3 | BBC | 5 | 2.5% HA | 0.8 | 0.4 |
| 5 | BBC | 5 | 5% HA | 2.5 | 2.2 |
| 6 | BBC | 5 | 2.5% Hylan-A | 2.7 | 2.6 |
| 8 | BBC | 5 | 5% Hylan-A | 0.3 | 0.5 |
| 10 | BBC | 5 | 30% Pluronic F-127 | 1.2 | 1.5 |
| 12 | BBC | 5 | 20% Pluronic F-127 | 4.3 | 1.6 |

### CONCLUSION

The presence of BBC was essential for the ability of rhBMP-2 loaded implants to induce bone formation.
Only formulations containing 20% Pluronic as a carrier induced good bone formation in a consistent manner.

### Example 12

### 08-2973

Study Objectives:
To compare two rat ectopic models: subcutaneous (SQ) versus intramuscular (IM) implantations
To evaluate different concentrations of OGF carriers

Study Design:

Twelve groups (n=3/group) of 4-5 week old male Long Evans rats received bilateral implants. Of these, six groups received bilateral subcutaneous implants in the chest (groups 1-6) and six groups received intramuscular implants in the back (groups 7-12). Two surgical implants (controls) contained 5 μg BMP-2 (signal) in 125 μl of glutamate buffer with 25 mg BBC (scaffold). Ten surgical implants contained 5 ng BMP-2 (signal) in 150 μl of varying carriers with 25 mg BBC (scaffold). Rats were sacrificed on day 14 post-implantation via CO₂ asphyxiation and the implants were harvested.

Details of the study design are outlined in Table 16 below.

TABLE 16
Study Design

| | | BMP-2 | | | |
|---|---|---|---|---|---|
| Group | | Dose | | Implant | Volume |
| # | Scaffold | [μg] | Carrier | Location | [μl] |
| 1 | BBC | 5 | no carrier | SQ | 125 |
| 2 | BBC | 5 | 2.5% HA | SQ | 150 |
| 3 | BBC | 5 | 20% Pluronic F-127 | SQ | 150 |
| 4 | BBC | 5 | 25% Pluronic F-127 | SQ | 150 |
| 5 | BBC | 5 | 30% Pluronic F-127 | SQ | 150 |
| 6 | BBC | 5 | 2.5% Hylan-A | SQ | 150 |
| 7 | BBC | 5 | no carrier | IM | 125 |
| 8 | BBC | 5 | 2.5% HA | IM | 150 |
| 9 | BBC | 5 | 20% Pluronic F-127 | IM | 150 |
| 10 | BBC | 5 | 25% Pluronic F-127 | IM | 150 |
| 11 | BBC | 5 | 30% Pluronic F-127 | IM | 150 |
| 12 | BBC | 5 | 2.5% Hylan-A | IM | 150 |

Evaluation:

The implants were harvested, fixed in 40% alcohol, embedded in methylmethacrylate, sectioned at approximately 5 microns and stained with hematoxylin and eosin (H&E) and toluidine blue.

Histopathologic analysis was performed by Kuber Sampath (Genzyme) and included semi-quantitative assessment of new bone production in the implant, using the scoring system outlined in Table 17.

TABLE 17
Scoring system for new bone production

| Score | Description |
|---|---|
| 0 | No new bone |
| 1 | 1-20% of the implant area contains new bone |
| | (w/o area occupied by BBC) |
| 2 | 21-40% of the implant area contains new bone |
| | (w/o area occupied by BBC) |
| 3 | 41-60% of the implant area contains new bone |
| | (w/o area occupied by BBC) |
| 4 | 61-80% of the implant area contains new bone |
| | (w/o area occupied by BBC) |
| 5 | 81-100% of the implant area contains new bone |
| | (w/o area occupied by BBC) |

Results:

Data are presented in Table 18 and FIG. 18.

TABLE 18
Table of histology scores

| | | | | Implant | Histology score | |
|---|---|---|---|---|---|---|
| Group # | Scaffold | BMP-2 Dose [μg] | Carrier | Location | Average | STDEV |
| 1 | BBC | 5 | no carrier | SQ | 5.0 | 0.0 |
| 2 | BBC | 5 | 2.5% HA | SQ | 3.2 | 1.6 |
| 3 | BBC | 5 | 20% Pluronic F-127 | SQ | 5.0 | 0.0 |
| 4 | BBC | 5 | 25% Pluronic F-127 | SQ | 4.2 | 1.6 |
| 5 | BBC | 5 | 30% Pluronic F-127 | SQ | 4.3 | 0.5 |
| 6 | BBC | 5 | 2.5% Hylan-A | SQ | 2.3 | 1.2 |
| 7 | BBC | 5 | no carrier | IM | 5.0 | 5.0 |
| 8 | BBC | 5 | 2.5% HA | IM | 3.8 | 3.8 |
| 9 | BBC | 5 | 20% Pluronic F-127 | IM | 5.0 | 5.0 |
| 10 | BBC | 5 | 25% Pluronic F-127 | IM | 5.0 | 5.0 |
| 11 | BBC | 5 | 30% Pluronic F-127 | IM | 5.0 | 5.0 |
| 12 | BBC | 5 | 2.5% Hylan-A | IM | 2.0 | 2.0 |

There was no difference in scores for new bone production between the two models.
Formulations having different Pluronic F-127 concentrations showed similar histology scores and were similar to controls.
Implants with 2.5% HA and Hylan A had lower histology scores compared to controls.

### Conclusions

Formulations implanted in subcutaneous and intramuscular implant sites showed comparable osteoinduction scores with all carriers.
All Pluronic carriers induced a similar level of bone formation as the positive control and two HA carriers induced less bone formation than the positive control.

### Example 13

### 09-4064

Study Objectives:
Effect of contrast agent
To evaluate polymer concentration
To evaluate carrier OGF ratios
To evaluate various carriers

Study Design:

Ten groups (n=4/group) of 4-5 week old male Long Evans rats received bilateral subcutaneous implants in the chest. Surgical implants had volume of 150 μl and containing 0 or 10 μg BMP-2 (signal), 25 mg of a bovine bone collagen (BBC, lot #17075-183, scaffold) and varying types of carriers.

Details of the study design are outlined in Table 19 below. All rats were sacrificed via CO₂ asphyxiation on day 14 and samples were harvested for analysis.

TABLE 19
Study Design

| Group | | BMP-2 Dose | |
|---|---|---|---|
| # | Scaffold | [μg] | Carrier |
| 1 | BBC | 0 | 22.5% Pluronic F-127 in PBS |
| | | | (negative control) |
| 2 | BBC | 10 | 22.5% Pluronic F-127 in PBS |
| | | | (positive control) |
| 3 | BBC | 10 | 22.5% Pluronic in 80% Isovue 370 |
| 4 | BBC | 10 | 22.5% Pluronic in 20% Isovue 370 |
| 5 | BBC | 10 | 30% Pluronic F-127 in PBS |
| 6 | BBC | 10 | 5% HA Mw 3 MDa/40% Pluronic |
| | | | F-127 |
| 7 | BBC | 10 | 2.5% HA Mw 3 MDa/20% Pluronic |
| | | | F-127 |
| 8 | BBC | 10 | Dermal Gel Extra (DGE) |
| 9 | BBC | 10 | PREVEL SILK |
| 10 | BBC | 10 | DGE/22.5% Pluronic F-127 |

Evaluation:

Samples were collected from each test site at the time of necropsy.

Each sample was cut into two pieces. One half of the sample was fixed in 10% formalin, embedded in paraffin, sectioned at approximately 5 microns and stained with hematoxylin and eosin (H&E).

Histopathologic evaluation included qualitative and semi-quantitative assessment of new cartilage and bone formation in the samples, using the scoring system outlined in Table 20. The distribution pattern of new bone/cartilage formation was also scored for each sample Table 21 (Lucy Phillips, B.V.Sc., A.C.V.P, Pathology Department, Genzyme Corporation).

TABLE 20
Scoring system for new bone/cartilage production

| Score | Description |
|---|---|
| 0 | No new bone |
| 1 | 1-25% of the implant contains new bone or cartilage |
| 2 | 25-50% of the implant contains new bone or cartilage |
| 3 | 50-75% of the implant contains new bone or cartilage |
| 4 | 75-100% of the implant contains new bone or cartilage |

TABLE 21
Distribution pattern of new bone/cartilage production

| Pattern | Description |
|---|---|
| A | Focal; new bone forms a rim around a cavitated center |
| B | Focal; new bone forms a rim around proliferating |
| | mesenchymal cells |
| C | Focal; new bone extends throughout the implant in |
| | a solid pattern |
| D | Multifocal; new bone forms a rim around multiple |
| | cavitated areas |
| E | Multifocal; new bone forms a rim around multiple |
| | foci of proliferating mesenchymal cells |
| F | Multifocal; several nodules of new bone extend |
| | throughout the implant in a solid pattern |

The other half of sample was cleaned of adherent tissue. The sample was placed in 2 ml of ice-cold 0.15 M NaCl/3 mM NaHCO₃ and then homogenized using a Polytron homogenizer. It was then centrifuged; the supernatant was decanted.

The residue was washed twice in 5 ml of 20 mM phosphate buffer, and then extracted in 5 ml of 0.6 NHCL overnight at 4° C. It was then centrifuged; the supernatant was decanted and sent for calcium analysis. Samples were analyzed on a Varian ICP-OES at 396 nm emission. (Martin Hanus, Department of Analytical Research and Development; Genzyme Corporation).

Results:

### Histopathologic Evaluation

The scatter plot graph of pathology scores is shown in FIG. 19.
Increasing the Pluronic F-127 concentration to 30% had no effect on the median new bone/cartilage scores (P>0.05)
The addition of 20% or 80% Isovue—370 to the 22.5% Pluronic F-'127 carrier had no effect on the median new bone/cartilage scores (P>0.05)
Adding 2.5% or 5% HA to the Pluronic F-127 tended to lower median new bone/cartilage scores, although this was not statistically significant (P>0.05)
DGE alone or added to the Pluronic F-127 tended to lower the median new bone/cartilage scores, although this was not statistically significant (P>0.05)
Prevelle silk tended to lower the median new bone/cartilage scores, although this was not statistically significant (P>0.05)
Groups containing Pluronic F-127, Pluronic F-127+Isovue 370 or Pluronic F-127+HA, tended to have either a “solid” distribution of bone throughout the implant or had a mixed pattern in which there where both solid areas (pattern C) and areas of rim formation around a central focus of proliferating mesenchymal cells (pattern B)
Groups containing DGE or Prevelle silk tended to form a rim of new bone around a central cavitated area containing BBC scaffold and DGE/Prevelle silk material (pattern D & E)

### Calcium Evaluation

Increased concentration of HA/F-127 in the carrier did not result in any differences in Ca concentration
The addition of 20% or 80% Isovue to the 22.5% Pluronic F-127 had no effect on the Ca concentration
Implants with Pluronic F-127 (groups 2-7 and 10) had higher calcium concentrations than implants without Pluronic F-127 (groups 8 & 9)

### CONCLUSIONS

Pluronic F-127 containing implants tended to have a predominantly solid distribution pattern of new bone/cartilage, whereas DGE and Prevelle silk implants frequently had a rim of new bone forming around a central cavitated area containing the scaffold and carrier
When HA or DGE was added to the Pluronic F-127, or when DGE or Prevelle

Silk was used as the sole carrier, new bone/cartilage production in implants trended lower
The addition of 20% or 80% Isovue to the 22.5% Pluronic F-127 carrier had no effect on osteoinduction potential
Implants with Pluronic F-127 (groups 2-7 and 10) had higher calcium concentrations than implants without Pluronic F-127 (groups 8 & 9)

### Example 14

### 09-4063

Study Objectives:

1. To evaluate various collagens

2. Effect of contrast agent

3. To evaluate cartilage particle size

4. To evaluate the effect of cartilage mass

5. To evaluate the OGF with Dermal Gel Extra (DGE) in the rat ectopic model

6. To evaluate various carriers

Study Design:

Twelve groups (n=4/group) of 4-5 week old male Long Evans rats received bilateral subcutaneous implants in the chest.
Ten groups received implants with 0-10 μg BMP-2 (signal), 150 μl of 22.5% Pluronic® F.-127 in PBS (carrier) and varying quantities and types of bone collagen (scaffold)
One group received implants with 3 μg BMP-2 (signal), 150 μl 22.5% Pluronic/80% Isovue 370 (carrier) and 25 mg BBC (scaffold).
One group received implants with 50 μg BMP-2 (signal) in 150 μl DGE (carrier).

Details of the study design are outlined in Table 22 below.

All rats were sacrificed via CO₂ asphyxiation on day 14 and samples were harvested for analysis.

TABLE 22
Study Design

| Group | | BMP-2 Dose | |
|---|---|---|---|
| # | Scaffold | [μg] | Carrier |
| 1 | BBC 70-425 um 25 mg | 0 | 22.5% Pluronic F-127 |
| 2 | BBC 70-425 um 25 mg | 3 | 22.5% Pluronic F-127 |
| 3 | BBC 70-425 um 25 mg | 10 | 22.5% Pluronic F-127 |
| 4 | BBC 70-425 um 25 mg | 3 | 22.5% Pluronic/80% |
| | | | Isovue 370 |
| 5 | BBC 70-250 um 10 mg | 3 | 22.5% Pluronic F-127 |
| 6 | BBC 70-250 um 10 mg | 10 | 22.5% Pluronic F-127 |
| 7 | BBC 70-250 um 25 mg | 3 | 22.5% Pluronic F-127 |
| 8 | BBC 70-250 um 25 mg | 10 | 22.5% Pluronic F-127 |
| 9 | BBC 70-250 um 25 mg | 0 | 22.5% Pluronic F-127 |
| 10 | RBC 70-425 um 25 mg | 3 | 22.5% Pluronic F-127 |
| 11 | RBC 70-425 um 25 mg | 10 | 22.5% Pluronic F-127 |
| 12 | — | 50 | DGE |

Evaluation:

Each sample was cut into two pieces. One half of the sample was fixed in 40% alcohol, embedded in methylmethacrylate, sectioned at approximately 5 microns and stained with hematoxylin and eosin (H&E).

Histopathologic evaluation included qualitative and semi-quantitative assessment of new cartilage and bone formation in the samples, using the scoring system outlined in Table 23. The distribution pattern of new bone/cartilage formation was also scored for each sample (Table 24) (Lucy Phillips, B.V.Sc., A.C.V.P, Pathology Department, Genzyme Corporation).

TABLE 23
Scoring system for new bone/cartilage production

| Score | Description |
|---|---|
| 0 | No new bone |
| 1 | 1-25% of the implant contains new bone or cartilage |
| 2 | 25-50% of the implant contains new bone or cartilage |
| 3 | 50-75% of the implant contains new bone or cartilage |
| 4 | 75-100% of the implant contains new bone or cartilage |

TABLE 24
Distribution pattern of new bone/cartilage production

| Pattern | Description |
|---|---|
| A | Focal; new bone forms a rim around a cavitated center |
| B | Focal; new bone forms a rim around proliferating |
| | mesenchymal cells |
| C | Focal; new bone extends throughout the implant in |
| | a solid pattern |
| D | Multifocal; new bone forms a rim around multiple |
| | cavitated areas |
| E | Multifocal; new bone forms a rim around multiple |
| | foci of proliferating mesenchymal cells |
| F | Multifocal; several nodules of new bone extend |
| | throughout the implant in a solid pattern |

The other half of sample was cleaned of adherent tissue. The sample was placed in 2 ml of ice-cold 0.15 M NaCl/3 mM NaHCO₃ and then homogenized using a Polytron homogenizer. It was then centrifuged; the supernatant was decanted.

The residue was washed twice in 5 ml of 20 mM phosphate buffer, and then extracted in 5 ml of 0.6 NHCL overnight at 4° C. It was then centrifuged; the supernatant was decanted and sent for calcium analysis. Samples were analyzed on a Varian ICP-OES; at 396 nm emission. (Martin Hanus, Department of Analytical Research and Development; Genzyme Corporation).

Results:

### Histopathologic Evaluation.

A scatter plot graph of the pathology scores is presented in FIG. 21.

There were no significant differences in median bone production scores between any of the treatment groups evaluated; however the following trends were notable:
For the 10 μg rhBMP-2 dose, implants containing 25 mg of the smaller particle BBC (group 8) showed a trend for greater new bone production relative to the dose-equivalent large particle BBC group (group 3, P>0.05) and the 10 mg small particle BBC group (group 6, P>0.05)
For implants containing 25 mg BBC+3 μg rhBMP-2+22.5% Pluronic F-127, addition of Isovue contrast agent did not significantly affect the production of new bone (P>0.05, group 4 versus group 2)
The rabbit bone collagen implants are a less robust model for evaluation of new bone production, as 3 and 10 μg groups (group 10 and 11) did not achieve statistical significance compared to the 0 μg control (group 9)
Rabbit bone collagen fragments were noted to be frequently larger than the 70<420 mm bovine bone fragments and this may have affected the amount of new bone production per implant
Implants containing 25 mg of BBC had a comparable median bone production score to implants containing 10 mg of BBC (groups 7&8 vs.5&6)
Although DGE implants without scaffold containing the highest dose of rhBMP-2 in this study, new bone production scores were variable. Furthermore, the distribution pattern of new bone in this group was also more variable than other groups

### Calcium Evaluation

At two doses of BMP-2 used in this study, BBC implants (group 2 and 3) and RBC implants (group 10 and 11) showed comparable Ca concentration. However, there was a trend for a higher Ca concentration for implants containing BBC versus RBC.

There were no differences in Ca concentration between any of the treatment groups evaluated.

### CONCLUSIONS

At two doses of BMP-2 used in this study, BBC and RBC implants showed comparable median bone production scores and Ca concentration. In the BBC and RBC-containing groups, new bone distribution tended to be in a focal to multifocal solid pattern
80% contrast agent in the carrier, 22.5% Pluronic, did not affect new bone production or Ca concentration
Changing scaffold mass did not affect new bone production or Ca concentration.
Implants containing smaller particle BBC showed a trend for greater new bone production relative to the dose-equivalent large particle BBC group
DGE group (which did not contain scaffold) new bone production scores were low and variable.

### Example 15

### 09-3469

Study Objectives:
To compare the osteogenic potential of two bone growth factors: rhBMP-2 and rhBMP-4
To evaluate various carriers
To evaluate the effect of surgical method (implantation versus injection) on the osteogenic potential of rhBMP-2 in the rat ectopic model

Study Design:

Twelve groups of 4-5 week old male Long Evans rats received bilateral subcutaneous implants in the chest. Surgical implants containing 0-3 μg BMP-2 (signal) in 150 μl 22.5% Pluronic F-127 in PBS, 2.5% HA/22.5% Pluronic F-127 in PBS or DGE, (carriers) and 25 mg of bovine bone collagen (BBC, lot #17075-114, scaffold).

Three groups of 4-5 week old male Long Evans rats received bilateral subcutaneous implants in the chest. Surgical implants containing 0.3-3 μg BMP-4 (signal) in 150 μl 22.5% Pluronic F-127 in PBS and 25 mg of BBC (scaffold).

Two groups of 4-5 week old male Long Evans rats were subcutaneously injected using a 14G or 20G needle. Surgical implants contained 3 μg BMP-2 (signal) in 150 22.5% Pluronic F-127 in PBS and 25 mg of BBC (scaffold).

All rats were sacrificed via CO₂ asphyxiation on day 14 and samples were harvested for analysis.

Details of the study design are outlined in Table 25 below.

TABLE 25
Study Design

| Group # | Scaffold | Signal | Carrier | Delivery | # Animals/Group |
|---|---|---|---|---|---|
| 1 | BBC | BMP-2, 3.0 μg | 22.5% Pluronic F-127 | Implant | 4 |
| 2 | BBC | BMP-2, 1.0 μg | 22.5% Pluronic F-127 | Implant | 4 |
| 3 | BBC | BMP-2, 0.3 μg | 22.5% Pluronic F-127 | Implant | 4 |
| 4 | BBC | BMP-2, 0.0 μg | 22.5% Pluronic F-127 | Implant | 4 |
| 5 | BBC | BMP-4, 3.0 μg | 22.5% Pluronic F-127 | Implant | 4 |
| 6 | BBC | BMP-4, 1.0 μg | 22.5% Pluronic F-127 | Implant | 4 |
| 7 | BBC | BMP-4, 0.3 μg | 22.5% Pluronic F-127 | Implant | 4 |
| 8 | BBC | BMP-2, 3.0 μg | DGE | Implant | 4 |
| 9 | BBC | BMP-2, 1.0 μg | DGE | Implant | 4 |
| 10 | BBC | BMP-2, 3.0 μg | 2.5% HA/22.5% Pluronic F-127 | Implant | 4 |
| 11 | BBC | BMP-2, 3.0 μg | 22.5% Pluronic F-127 | Injection | 4 |
| 12 | BBC | BMP-2, 3.0 μg | 22.5% Pluronic F-127 | Injection | 6 |

Evaluation:

Samples were collected from each test site at the time of necropsy.

Implants were fixed in 10% neutral buffered formalin. Tissues were decalcified, routinely processed, embedded in paraffin, sectioned at 5 microns and stained with hematoxylin and eosin (H&E), and toluidine blue for light microscopic evaluation.

Histopathologic evaluation included qualitative and semi-quantitative assessment of new cartilage and bone formation in the samples and used the scoring system outlined in Table 26. The distribution pattern of new bone/cartilage formation was also scored for each sample (Table 27) (Lucy Phillips, B.V.Sc., A.C.V.P, Pathology Department, Genzyme Corporation).

TABLE 26
Scoring system for new bone/cartilage

| Score | Description |
|---|---|
| 0 | No new bone |
| 1 | 1-25% of the implant contains new bone or cartilage |
| 2 | 25-50% of the implant contains new bone or cartilage |
| 3 | 50-75% of the implant contains new bone or cartilage |
| 4 | 75-100% of the implant contains new bone or cartilage |

TABLE 27
Distribution pattern of new bone/cartilage

| Pattern | Description |
|---|---|
| A | Focal; new bone forms a rim around a cavitated center |
| B | Focal; new bone forms a rim around proliferating |
| | mesenchymal cells |
| C | Focal; new bone extends throughout the implant in |
| | a solid pattern |
| D | Multifocal; new bone forms a rim around multiple |
| | cavitated areas |
| E | Multifocal; new bone forms a rim around multiple |
| | foci of proliferating mesenchymal cells |
| F | Multifocal; several nodules of new bone extend |
| | throughout the implant in a solid pattern |

Results:

### Histopathologic Evaluation

The scatter plot graph of pathology scores is shown in FIG. 24.
There was a dose-responsive increase in new bone and cartilage production within surgical implants loaded with 0-3 μg of rhBMP-2
There was minimal bone production within rhBMP-4 loaded scaffolds, irrespective of the dose
Median scores for bone/cartilage production were highest for surgical implants containing 3 μg rhBMP-2 delivered with Pluronic or HA/Pluronic
Implants with DGE had a slightly lower median score, and frequently resulted in a rim of bone production around a cavitated center containing the DGE and BBC scaffold
Median scores for bone/cartilage production tended to be slightly higher for rhBMP-2 delivered by surgical implantation compared to subcutaneous injection
Furthermore, surgical implantation gave a predominantly diffuse distribution of the new bone, while subcutaneous injection resulted in a variable distribution pattern (diffuse, or formation of a rim of new bone around a cavitated or cellular center)

### CONCLUSIONS

There was a dose responsive increase in histology score for implants with 0-3 μg of rhBMP-2 loaded on BBC with Pluronic F-127.
There was minimal bone production within rhBMP-4 loaded scaffolds, irrespective of the dose.
Scaffolds with 3 μg rhBMP-2 and Pluronic F-127 or HA/Pluronic F-127 had a non-statistical trend for more bone/cartilage production compared to those with DGE
Implants with Pluronic F-127 or HA/Pluronic F-127 had a diffuse distribution of bone throughout the implant, whereas DGE had a rim of new bone around a cavitated center.
There was a non-statistical trend for slightly more bone/cartilage production with surgical implantation rather than subcutaneous injection. Furthermore, subcutaneous injection resulted in a variable distribution pattern.

A number of embodiments of the invention have been described. Nevertheless, it will be understood that various modifications may be made without departing from the spirit and scope of the invention.

What is claimed is:

1. An injectable composition comprising an osteogenic growth factor (OSF), a reverse thermo-sensitive biodegradable poloxamer, and type I collagen in an aqueous vehicle.

2. The composition of claim 1 in which the OSF is a bone morphogenetic protein (BMP).

3. The composition of claim 2 in which the BMP is a homodimer of BMP-2 or BMP-4 or BMP-6 or BMP-7 (OP-1) or a BMP-2/7 hetrodimer or a combination of selected BMPs.

4. The composition of claim 1 in which the BMP is present at a concentration of less than 3.5 mg per g of collage-poloxamer scaffold.

5. The composition of claim 1 further comprising a mineral.

6. The composition of claim 5 in which the mineral is tricalcium phosphate or synthetic or natural hydroxylapatite.

7. The composition of claim 1 further comprising a hyaluronic compound.

8. The composition of claim 7 in which the hyaluronic compound has a molecular weight >500Da.

9. The composition of claim 1 or claim 7 in which the hyaluronic compound is cross-linked hyaluronic acid.

10. The composition of claim 1 in which the composition viscosity at room temperature is suitable for injection from a syringe.

11. The composition of claim 10 in which the composition exhibits a syringe extrusion force <30 Newtons.

12. The composition of claim 1 in which the composition has between 50 and 80% liquid by weight.

13. The composition of claim 1 in which the average particle size of the collagen is between 70 to 425 μm.

14. The composition of claim 1 in which the composition is a malleable putty.

15. The composition of claim 1 in which the aqueous vehicle is a buffered solution in which the OSF, the type I collagen and the polymer are dissolved and/or suspended.

16. The composition of claim 1 in which the composition further includes a radio-contrast agent.

17. The composition of claim 1 in which the composition is a lyophilized mixture.

18. The composition of claim 1 comprising a glycosaminoglycan.

19. The compound of claim 18 in which the glycosaminoglycan is chondroitin sulfate or chitosan.

20. A method of treating a patient in need thereof, comprising injecting the composition of claim 1 at a site where bone growth is desired.

21. The method of claim 20 in which the site is a site where the patient has undergone a spinal fusion.

22. The method of claim 20 which the site is a bone fracture site.

23. A method of making a medicament for treating spinal fusion or a bone fracture site, said medicament comprising the composition of claim 1.

24. A medicament for effecting bone growth, said medicament comprising the composition of claim 1.
